(19) United States
(12) Patent Application Publication
(10) Pub. No.: US 2011/0251493 A1
(43) Pub. Date: Oct. 13, 2011

(54) METHOD AND SYSTEM FOR MEASUREMENT OF PHYSIOLOGICAL PARAMETERS

(75) Inventors: Ming-Zher Poh, Cambridge, MA (US); Daniel J. McDuff, Cambridge, MA (US); Rosalind W. Picard, Newtonville, MA (US)

(73) Assignee: Massachusetts Institute of Technology, Cambridge, MA (US)

(21) Appl. No.: 13/048,965

(22) Filed: Mar. 16, 2011

Related U.S. Application Data
(60) Provisional application No. 61/316,047, filed on Mar. 22, 2010.

(51) Int. Cl. A61B 6/00; G06K 9/00

(52) U.S. Cl. 600/477; 382/128

(57) ABSTRACT

Method and system for measuring physiological parameters. The method includes capturing a sequence of images of a human face and identifying the location of the face in a frame of the video and establishing a region of interest including the face. Pixels are separated in the region of interest in a frame into at least two channel values forming raw traces over time. The raw traces are decomposed into at least two independent source signals. At least one of the source signals is processed to obtain a physiological parameter.

This application claims priority to provisional application Ser. No. 61/316,047 filed Mar. 22, 2010, the contents of which are incorporated herein by reference in their entirety.

### BACKGROUND OF THE INVENTION

This invention relates to measurement of physiological parameters and more particularly to a simple, low-cost method for measuring multiple physiological parameters using digital color video.

The option of monitoring a patient's physiological signals via a remote, non-contact means has promise for improving access to and enhancing the delivery of primary healthcare. Currently, proposed solutions for non-contact measurement of vital signs such as heart rate (HR) and respiratory rate (RR) include laser Doppler [1], microwave Doppler radar [2] and thermal imaging [3, 4]. The numbers in brackets refer to the references included herewith, the contents of all of which are incorporated herein by reference. Non-contact assessment of heart rate variability (HRV), an index of cardiac autonomic activity [5], presents a greater challenge and few attempts have been made [6-8]. Despite these impressive advancements, a common drawback of the above methods is that the systems are expensive and require specialist hardware.

Photoplethysmography (PPG) is a low-cost and noninvasive means of sensing a cardiovascular blood volume pulse (BVP) through variations in transmitted or reflected light [9]. Although PPG is typically implemented using dedicated light sources (e.g., red and/or infra-red wavelengths), Verkruysse et al. showed that pulse measurements from the human face are attainable with normal ambient light as the illumination source [10]. However this study lacked rigorous physiological and mathematical models amendable to computation; it relied instead on manual segmentation and heuristic interpretation of raw images with minimal validation of performance characteristics.

### SUMMARY OF THE INVENTION

According to a first aspect, the invention is a method for measuring physiological parameters. The method includes capturing a sequence of images of a human face and identifying the location of the face in a frame of the captured images and establishing a region of interest including the face or a subset thereof. Pixels in the region of interest are separated into at least two channel values forming raw traces over time. The raw traces are decomposed into at least two independent source signals. At least one of the source signals is processed to obtain a physiological parameter.

In an embodiment of this aspect of the invention, the pixels are spatially averaged in the region of interest to yield a measurement point for each of the at least two channel values for each frame. This embodiment may include detrending and normalizing the raw traces.

In another preferred embodiment of this aspect of the invention the identifying location step utilizes a boosted cascade classifier. In this embodiment, the region of interest is a box drawn around the face or a subset thereof. The traces may be approximately five seconds to fifteen minutes long. In a preferred embodiment, the detrending step is applied to the raw traces. The raw traces are normalized and in a preferred embodiment the decomposing step uses independent component analysis.

In another preferred embodiment of this aspect of the invention the processing step includes smoothing and filtering of the separated source signals. In preferred embodiments, the physiological parameters include the blood volume pulse, cardiac interbeat interval, heart rate, respiration rate or heart rate variability. It is preferred that the video be color video. The capturing step utilizes a digital camera, web cam or mobile phone camera. In a preferred embodiment, the spatially averaging step computes a spatial mean, median or mode. The heart rate variability may be determined by power spectral density estimation. Simultaneous physiological measurements may be made of multiple users.

In yet another aspect, the invention is a method for automatic measurement of physiological parameters of at least one subject from video of a body part of the subject. The method includes localization of a region of interest from frames of the video and extraction of input signals from the region of interest. The input signals are blind source separated to recover separated source signals. One or more of the separated source signals is selected and the one or more selected source signals is processed to provide a measurement of the physiological parameters. In a preferred embodiment of this aspect of the invention, the body part is a face or a subset thereof. The localization step may be based on a trained classifier.

In a preferred embodiment, the extraction of input signals from the region of interest include separating red, green and blue channels and computing a spatial mean, median or mode of these channels for each video frame. The blind source separation may include detrending and normalizing the input signals extracted from the region of interest. It is preferred that the blind source separation incorporate independent component analysis for the separation of source signals from the detrended and normalized input signals. The separated source signals may be processed in a time window on the order of five seconds to fifteen minutes. It is also preferred that the processing of the one or more selected source signals includes moving average filtering to obtain a blood volume pulse. In this aspect of the invention, the physiological parameters include heart rate, respiratory rate and heart rate variability.

In still another aspect, the invention is a system for determining physiological parameters, including a camera for capturing video of a human face to generate at least two signals and a computer running a program operating on the signals to determine the blood volume pulse from which other physiological parameters may be determined.

The present invention thus provides a simple, low-cost method for measuring multiple physiological parameters using a basic web cam or other color digital video camera. High degrees of agreement were achieved between the measurements across all physiological parameters. The present invention has significant potential for advancing personal healthcare and telemedicine.

### BRIEF DESCRIPTION OF THE DRAWING

FIG. 1a is a photograph of a human face within a video frame.

FIG. 1b are decompositions of the face in FIG. 1a decomposed into red, green and blue channels.

FIG. 1c are red, green and blue raw signals.

FIG. 1d is a schematic representation showing independent component analysis applied to the separate three independent source signals.

FIG. 1e are graphs of the separated source signals.

FIG. 2a are plots of a blood volume pulse waveform using the present invention in comparison with a waveform detected by a finger BVP sensor. The selected source signal was smoothed using a five-point moving average filter and bandpass filtered, 0.7 to 4 Hz.

FIG. 2b are plots of interbeat intervals formed by extracting the peaks from the BVP waveforms according to an embodiment of the invention and with a finger BVP sensor.

FIG. 2c illustrates a normalized Lomb periodogram of the detrended interbeat intervals exhibiting a dominant HF component.

FIGS. 2d-2f are an example recording exhibiting a dominant LF component.

FIG. 3a is a plot of an interbeat interval series from a webcam.

FIG. 3b is a plot showing a normalized Lomb periodogram showing HF power (0.15-0.4 Hz) centered at 0.23 Hz.

FIG. 3c is a plot of respiration signal versus time showing a respiration waveform measured by a chest belt sensor.

FIG. 3d is a plot of normalized power versus frequency showing a normalized Lomb periodogram showing the fundamental respiration frequency of 0.23 Hz.

FIG. 4a is a scatter plot comparing measurements of heart rate.

FIG. 4b is a scatter plot comparing measurements of high frequency power.

FIG. 4c is a scatter plot comparing measurements of low frequency power.

FIG. 4d is a scatter plot comparing measurements of the ratio of low frequency power to high frequency power.

FIG. 4e is a scatter plot comparing measurements of respiration rate between a web cam an reference sensors (finger BVP for HR and HRV measurements, chest belt respiration sensor for respiration rate).

FIG. 5 is a flow chart describing an embodiment of the method of the invention.

### DESCRIPTION OF THE PREFERRED EMBODIMENT

Recently, the inventors herein developed a robust method for automated computation of heart rate from digital color video recordings of the human face [11]. In this patent application we extend the methodology to quantify multiple physiological parameters. Specifically, the invention disclosed herein extracts the blood volume pulse for computation of heart rate, respiration rate as well as heart rate variability.

First of all, some of the theory on which the present invention is based will now be provided. Independent component analysis (ICA) is a relatively new technique for uncovering independent signals from a set of observations that are composed of linear mixtures of the underlying sources [12]. The underlying source signal of interest in this patent application is the blood volume, pulse that propagates throughout the body. During the cardiac cycle, volumetric changes in the facial blood vessels modify the path length of the incident ambient light such that the subsequent changes in amount of reflected light indicate the timing of cardiovascular events. By capturing a sequence of images of the facial region with a webcam, the red, green and blue (RGB) color sensors pick up a mixture of reflected plethysmographic signal along with other sources of fluctuations in light due to artifacts. Given that hemoglobin absorptivity differs across the visible and near-infrared spectral range [13], each color sensor records a mixture of the original source signals with slightly different weights. These observed signals from the RGB color sensors are denoted by y₁(t), y₂(t) and y₃(t) respectively, which are amplitudes of the recorded signals at time point t. We assume three underlying source signals, represented by x₁(t), x₂(t) and x₃(t). The ICA model assumes that the observed signals are linear mixtures of the sources, that is, y(t)=Ax(t) where the column vectors y(t)=|y₁(t), y₂(t), y₃(t)|^{T}, x(t)=[x₁(t), x₂(t), x₃(t)]^{T} and the square 3×3 matrix A contains the mixture coefficients aᵢⱼ. The aim of ICA is to find a demixing matrix W that is an approximation of the inverse of the original mixing matrix A whose output \hat{x}(t)=Wy(t) is an estimate of the vector x(t) containing the underlying source signals. To uncover the independent sources, W must maximize the non-Gaussianity of each source. In practice, iterative methods are used to maximize or minimize a given cost function that measures non-Gaussianity.

The technology disclosed herein has been evaluated at the Massachusetts Institute of Technology. Experiments included 12 participants of both genders (four females), different ages (18-31 years) and skin color. The experiments were conducted indoors and with a varying amount of ambient sunlight entering through windows as the only source of illumination. Participants were seated at a table in front of a laptop computer at a distance of approximately 0.5 m from a built in webcam (iSight camera). During the experiments, participants were asked to keep still, breathe spontaneously and face the webcam while their video was recorded for one minute. All videos were captured in color (24-bit RGB with three channels with 8 bits/channel) at 15 frames per second with pixel resolution of 640×480, and saved in AVI format on the laptop computer. We also recorded the blood volume pulse of the participants along with spontaneous breathing using an FDA-approved finger BVP sensor and chest belt respiration sensor (Flexcomp Infiniti by Thought Technologies Limited) respectively at a sampling rate of 256 Hz.

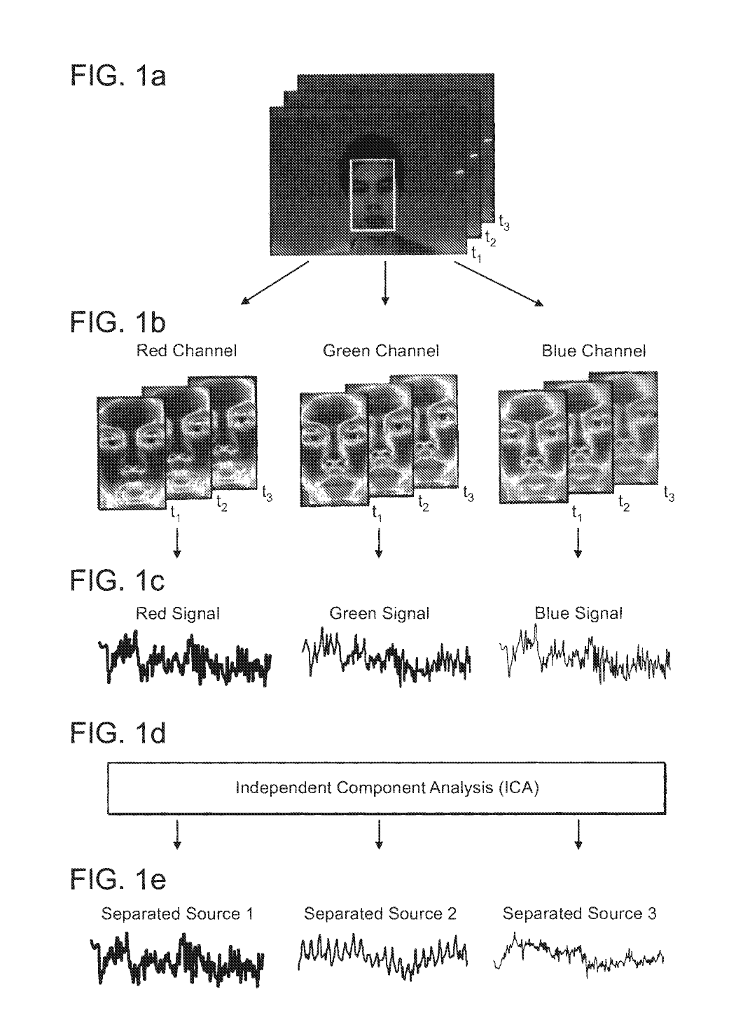

All of the video and physiological recordings were analyzed offline using software written in MATLAB. With reference now to FIG. 1, this figure provides an overview of the stages involved in the present approach to recovering the blood volume pulse from the webcam videos. We utilized the Open Computer Vision library [14] to automatically identify the coordinates of the face location in the first frame of the video recording using a boosted cascade classifier [15]. The algorithm returned the x- and y-coordinates along with the height and width that define a box around the face. We selected the center 60% width and full height of the box as the region of interest (ROI) for subsequent calculations.

The region of interest was then separated into three RGB channels, as shown in FIG. 1b, and spatially averaged over all pixels in the region of interest to yield a red, blue and green respectively. Each trace was one minute long. The raw traces were detrended using a procedure based on a smoothness priors approach [16] with the smoothing parameter λ=10 (cut-off frequency of 0.89 Hz) and normalized as follows:

$${y}_{i}^{′} ( t ) = \frac{{y}_{i} ( t ) - {μ}_{i}}{{σ}_{i}}$$

for each i=1, 2, 3, and where μᵢ and σᵢ are the mean and standard deviation of yᵢ(t) respectively. The normalized raw traces are then decomposed into three independent source signals using ICA (FIG. 1d) based on the joint approximate diagonalization of eigenmatrices (JADE) algorithm [17]. Independent component analysis is able to perform motion-artifact removal by separating the fluctuations caused predominantly by the blood volume pulse from the observed raw signals [11]. However, the order in which the ICA returns the independent components is random. Thus, the component whose power spectrum contained the highest peak was then selected for further analysis.

The separated source signal was smoothed using a five-point moving average filter and bandpass filtered (128-point Hamming window, 0.7 to 4 Hz). To refine the BVP peak fiducial point, the signal was interpolated with a cubic spline function at a sampling frequency of 256 Hz. We developed an algorithm to detect the BVP peaks in the interpolated signal and applied it to obtain the interbeat intervals (IBIs). To avoid inclusion of artifacts such as ectopic beats or motion, the IBIs were filtered using the NC-VT (non-causal of variable threshold) algorithm [18] with a tolerance of 30%. Heart rate was calculated from the mean of the IBI time series as 60/ I̅B̅I̅.

Analysis of the heart rate variability was performed by power spectral density (PSD) estimation using the Lomb periodogram. The low frequency power (LF) and high frequency power (HF) were measured as the area under the PSD curve corresponding to 0.04-0.15 Hz and 0.15-0.4 Hz respectively and quantified in normalized units to minimize the effect on the values of the changes in total power. The LF component is modulated by baroreflex activity and includes both sympathetic and parasympathetic influences [19]. The HF component reflects parasympathetic influence on the heart through efferent vagal activity and is connected to 230 respiratory sinus arrhythmia, a cardiorespiratory phenomenon characterized by interbeat interval fluctuations that are in phase with inhalation and exhalation. We also calculated the LF/HF ratio considered to mirror sympatho/vagal balance or to reflect sympathetic modulations.

Since the HF component is connected with breathing, the respiration rate can be estimated from the HRV power spectrum. When the frequency of respiration changes, the center frequency of the HF peak shifts in accordance with the respiration rate [20]. Thus, we calculated respiration rate from the center frequency of the HF peak f_{Hf Peak} in the heart rate variability power spectral density plot derived from the webcam recordings as 60/f_{Hf Peak}. The respiratory rate measured using the chest belt sensor was determined by the frequency corresponding to the dominant peak f_{resp peak} in the power spectral density plot of the recorded respiratory wave form using 60/f_{resp peak}.

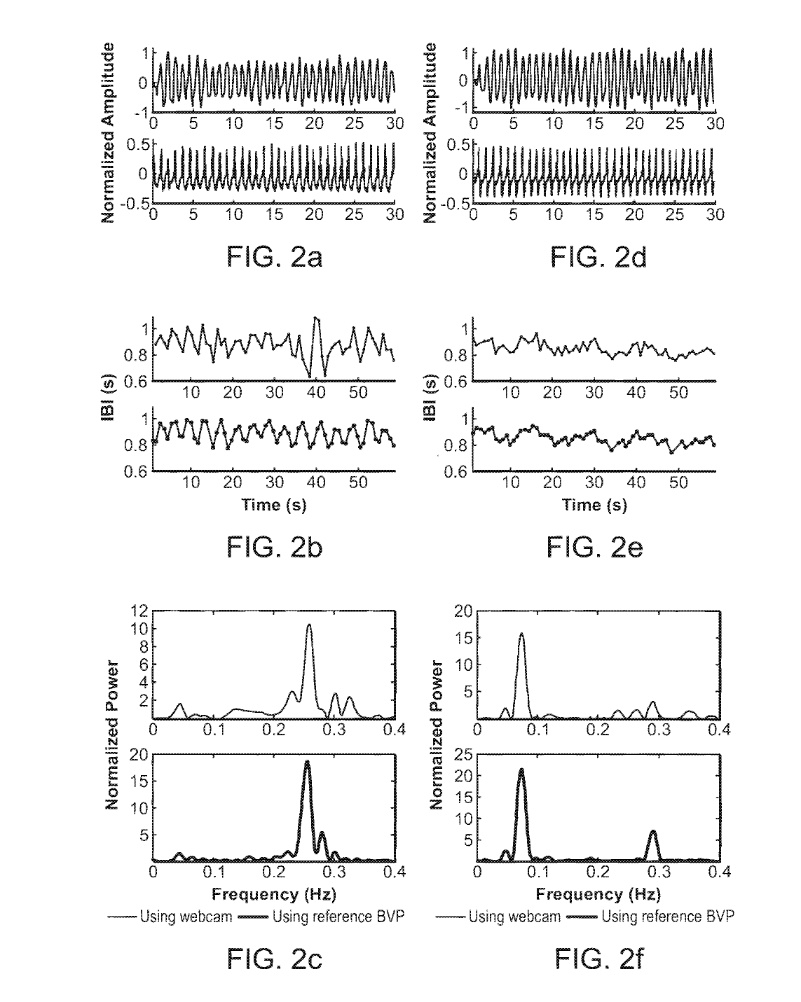

Using the techniques set forth above, we extracted the blood volume pulse waveforms from the webcam recordings via ICA. A typical example of the recovered BVP recordings is shown in FIG. 2a along with the BVP recorded with a Flexcomp sensor. It is evident that the two signals are in close agreement and their respective IBI signals are comparable (FIG. 2b). Since the IBI series is irregularly time-sampled, we utilized the Lomb periodogram to obtain the PSD to avoid resampling and inferring probable replacement values for excluded samples. The resulting spectra are presented in FIG. 2c. Both spectra are comparable and exhibit a dominant HF component. A second example of HRV assessment is shown in FIGS. 2d-f. Once again, the BVP and the IBI signals are similar and the HRV power spectra both exhibit a dominant LF component.

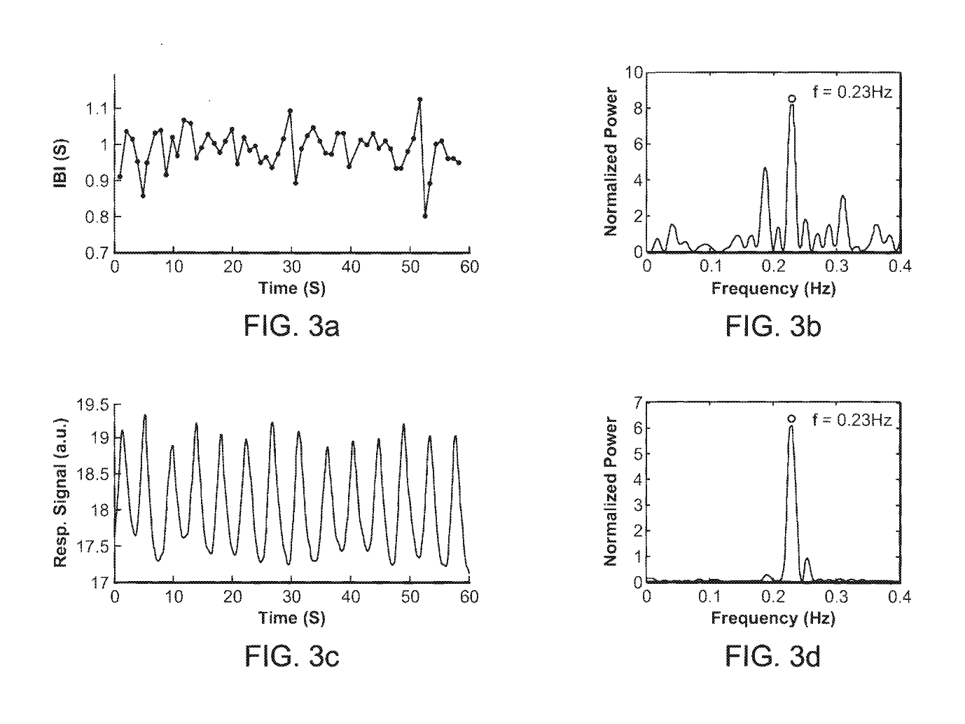

We were able to determine RR from the HRV power spectrum by locating the center frequency of the HF peak. FIG. 3a presents an IBI time series and its corresponding PSD (FIG. 3b). The center frequency of the HF peak was 0.23 Hz (14 breaths/min) and corresponds to the fundamental breathing rate computed from the PSD (FIG. 3d) of the measured respiratory signal using a chest belt sensor (FIG. 3c).

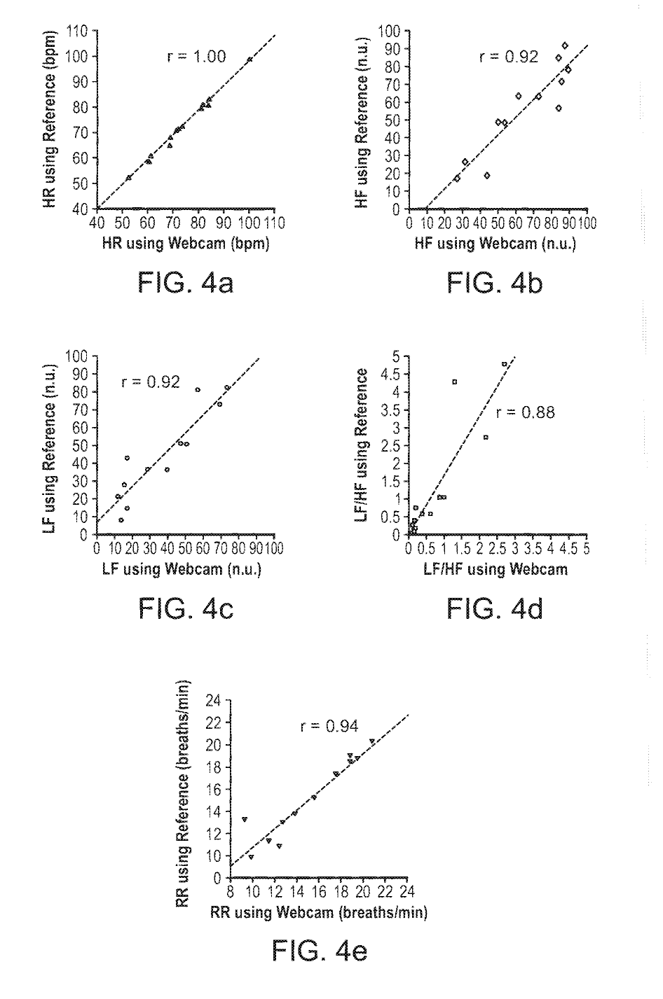

The level of agreement between the physiological measurements made by the invention disclosed herein and by reference sensors was accessed using Pearson's correlation coefficients (n=12). Correlation scatter plots for each measured parameter are shown in FIG. 4. The webcam-derived physiological measurements were strongly correlated across all parameters with r=1.0 for HR, r=0.92 for HF and LF, r=0.88 for LF/HF and r=0.94 for RR (p≧0.001 for all). The root-mean squared error of HR, HF, LF, LF/HF, RR was 1.24 bpm, 12.3 n.u., 12.3 n.u., 1.1, 1.28 breaths/min respectively. The results of the present studies are shown in Table 1.

TABLE 1
SUMMARY OF OVERALL RESULTS

| | Heart | Respiratory | Heart Rate Variability | | |
|---|---|---|---|---|---|
| | Rate | Rate | LF | HF | |
| Statistic | (bpm) | (breaths/min) | (n.u.) | (n.u.) | LF/HF |
| Mean error | 0.95 | 0.12 | 7.53 | 7.53 | 0.57 |
| SD of error | 0.83 | 1.33 | 10.17 | 10.17 | 0.98 |
| RMSE | 1.24 | 1.28 | 12.3 | 12.3 | 1.1 |
| Correlation | 1.00 | 0.94 | 0.92 | 0.92 | 0.88 |
| coefficient | | | | | |

All analyses performed on one-minute recordings from 12 participants.

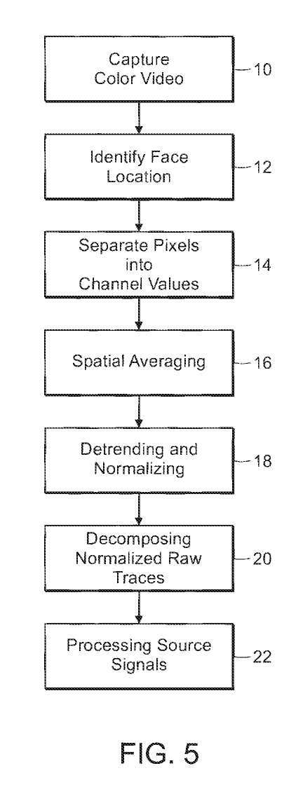

The steps of the method of an embodiment of the invention disclosed herein are shown in FIG. 5. In step 10, color video of the human face is captured. The location of the face is identified in step 12 along with establishing a region of interest including the face. Pixels in the region of interest are separated into three channel values at step 14 and spatially averaged over all pixels in the region of interest at step 16 to form raw traces. The raw traces are detrended and normalized at step 18. The normalized raw traces are decomposed into independent source signals at 20 and at least one of the source signals is processed to obtain a physiological parameter at step 22.

On the basis of the results in Table 1, we demonstrated the feasibility of using a simple webcam to measure multiple physiological parameters. These parameters include vital signs such as heart rate and respiration rate, as well as correlates of cardiac autonomic function through heart rate variability. Our data demonstrate that there is a strong elation between these parameters derived from the webcam recordings and standard reference sensors. Regarding the choice of measurement epoch, a recording of 1-2 minutes is needed to assess the spectral components of HRV [5] and an averaging period of 60 beats improves the confidence in the single timing measurement from the BVP waveform [9]. The face detection algorithm is subject to head rotation limits. About three axes of pitch, rotation and yaw, the limits were 32.6±4.84, 33.4±2.34 and 18.6±3.75 degrees from the frontal position.

The results set forth above should be considered in light of limitations of the present study. First of all, the webcam video sampling rate fluctuated around 15 fps due to the use of a standard PC for image acquisition, causing misalignment of the BVP peaks compared to the reference signal. The performance of the present invention can be boosted if each video frame were time stamped and the signals were resampled. Performance can also be boosted by (1) using a camera with a higher frame rate or one dedicated to this computation, or by (2) using multiple slow (e.g. 30 fps) cameras, slightly uttered in their time sampling synchronization offsets so that their measures may be combined to get higher temporal resolution. Second, the video sampling rate is much lower than recommended rates (greater than or equal to 250 Hz) for HRV analysis. By interpolating at 256 Hz to reline the peaks in the BVP and improve timing estimations we achieved the high correlation shown in Table 1 above. The PPG beat-to-beat variability can be affected by changes in the pulse transit time, which is related to arterial compliance and blood pressure, but it has been shown to be a good surrogate of HRV at rest [21]. A limitation of the system disclosed herein is that only three source signals can be recovered. However, our results suggest that this is sufficient to obtain accurate measurements of the BVP.

It is recognized that modifications and variations of the invention disclosed herein will be apparent to those of ordinary skill in the art, and it is intended that all such modifications and variations be included within the scope of the appended claims.

### REFERENCES

[1] S. Ulyanov and V. Tuchin, “Pulse-wave monitoring by means of focused laser beams scattered by skin surface and membranes,” in Proc SPIE, Los Angeles, Calif., USA, 1884, pp. 160-167.
[2] E. Greneker, “Radar sensing of heartbeat and respiration at a distance with applications of the technology,” in Proc Conf RADAR, Edinburgh, UK, 1997, pp, 150-154.
[3] M. Garbey, N. Sun, A. Merla, and I. Pavlidis, “Contact-free measurement of cardiac pulse based on the analysis of thermal imagery,” IEEE Trans Biomed Eng, vol. 54, pp. 1418-26, August 2007.
[4] J. Fei and L Pavlidis, “Thermistor at a Distance: Unobtrusive Measurement of Breathing,” IEEE Trans Blamed Eng, vol. 57, pp. 988-998, 2009.

[5] M. Malik, J. Rigger, A. Camm, R, Kleiger, A. Malliani, A. Moss, and P. Schwartz, “Heart rate variability: Standards of measurement, physiological interpretation, and clinical use,” Eur Heart J, vol. 17, p. 354, 1996.
[6] S. Suzuki, T. Matsui, S. Gotoh, Y. Mori, 5. Takase, and M. Ishihara, “Development of Non-contact Monitoring System of Heart Rate Variability (HRV)—An Approach of Remote Sensing for Ubiquitous Technology,” in Proc Int Conf Ergonomics and Health Aspects of Work with Computers, San Diego, Calif., 2009, p. 203.
[7] G. Lu, F. Yang, Y. Tian, X. Jing, and J. Wang, “Contact-free Measurement of Heart Rate Variability via a Microwave Sensor,” Sensors, vol. 9, pp. 9572-9581, 2009,
[8] U. Morbiducci, L. Scalise, M. Dv Melis, and M. Grigioni, “Optical vibrocardiography: a novel tool for the optical monitoring of cardiac activity,” Ann Biomed Eng, vol. 35, pp. 45-58, January 2007.
[9] J. Allen, “Photoplethysmography and its application in clinical physiological measurement,” Physiol Meas, vol. 28, pp. R1-39, March 2007.
[10] W. Verkruysse, L. O. Svaasand, and J. S. Nelson, “Remote plethysmographic imaging using ambient light,” Opt Express, vol 16, pp. 21434-45, Dec. 22, 2008.
[11] M. Z. Poh, D. J. McDuff, and R. W. Picard, “Non-contact, automated cardiac pulse measurements using video imaging and blind source separation,” Opt Express, vol. 18, pp. 10762-74, May 7, 2010.
[12] P. Comon, “Independent component analysis, a new concept?,” Signal Process, vol. 36, pp. 287-314, 1994.
[13] W. G. Zijlstra, A. Buursma, and W. P. Meeuwscn-van der Roest, “Absorption spectra of human fetal and adult oxyhemoglobin, deoxyhemoglobin, carboxyhemoglobin, and methemoglobin,” Clin Chem, vol, 37, pp. 1633-8, September 1991.
[14] A. Noulas and B. Kröse, “EM detection of common origin of multimodal cues,” in Proc ACM roof Multimodal Interfaces, 2006, pp. 201-208.
[15] P. Viola and M. Jones, “Rapid object detection using a boosted cascade of simple features” in Proc IEEE Conf Computer Vision and Pattern Recognition, 2001, p. 511.
[16] M. P. Tarvainen, P. O. Ranta-Aho, and P. A. Karjalainen, “An advanced detrending method with application to HRV analysis,” IEEE Trans Biomed Eng, vol. 49, pp. 172-5, February 2002.
[17] J.-F. Cardoso, “High-order contrasts for independent component analysis,” Neural Comput, vol. 11, pp. 157-192, 1999.
[18] J. Vila, F. Palacics, J. Presedo, M. Femandez-Delgado, P. Felix, and S. Barro, “Time-frequency analysis of heart-rate variability,” IEEE Eng Med Biol Mag, vol. 16, pp. 119-26, September-October 1997.
[19] S. Akselrod, D. Gordon, F. A. Ubel, D. C. Shannon, A, C. Berger, and R. J. Cohen, “Power spectrum analysis of heart rate fluctuation: a quantitative probe of beat-to-beat cardiovascular control,” Science, vol. 213, pp. 220-2, Jul. 10, 1981.
[20] T. Brown, L. Beightol, J. Koh, and D. Eckberg, “Important influence of respiration on human RR interval power spectra is largely ignored,” J Appl Physiol, vol, 75, p. 2310, 1993,
[21] E. Gil, M. Orini, R. BailÛn, j, Vergara, L. Mainardi, and P. Laguna, “Photoplethysmography pulse rate variability as a surrogate measurement of heart rate variability during non-stationary conditions,” Physiol Meas, vol. 31, pp. 1271-1290, 2010.

What is claimed is:

1. Method for measuring physiological parameters comprising:
capturing a sequence of images of a human face;
identifying location of the face in a frame of the captured images and establishing a region of interest including the face;
separating pixels in the region of interest in a frame into at least two channel values forming raw traces over time;
decomposing the raw traces into at least two independent source signals; and
processing at least one of the source signals to obtain a physiological parameter.

2. The method of claim 1 further including spatially averaging over all pixels in the region of interest to yield a measurement point for each of the at least two channel values for each frame.

3. The method of claim 1 further including detrending and/or normalizing the raw traces.

4. The method of claim 1 wherein the identifying location step utilizes a boosted cascade classifier.

5. The method of claim 1 wherein the region of interest is a box drawn around the face or a subset thereof.

6. The method of claim 1 wherein the traces are approximately five seconds to fifteen minutes long.

7. The method of claim 1 wherein the decomposing step uses independent component analysis.

8. The method of claim 1 wherein the processing step includes filtering the separated source signals.

9. The method of claim 1 wherein the physiological parameter is blood volume pulse.

10. The method of claim 1 wherein the physiological parameter is cardiac interbeat interval.

11. The method of claim 1 wherein the physiological parameter is heart rate.

12. The method of claim 1 wherein the physiological parameter is heart rate variability.

13. The method of claim 1 wherein the physiological parameter is respiration rate.

14. The method of claim 1 wherein the video is color video.

15. The method of claim 1 wherein the capturing step utilizes a digital camera, webcam or mobile phone camera.

16. The method of claim 2 wherein the spatially averaging step computes a spatial mean, median or mode.

17. The method of claim 12 wherein heart rate variability is determined by taking a function of the results of power spectral density estimation.

18. The method of claim wherein simultaneous physiological measurements of multiple users is obtained.

19. Method for automatic measurement of physiological parameters of at least one subject from video of a body part of the subject comprising:
localization of a region of interest from frames of the video;
extraction of input signals from the region of interest;
blind source separation of the input signals to recover separated source signals;
selection of one or more of the separated source signals; and
processing the one or more selected source signals to provide a measurement of the physiological parameters.

20. The method of claim 19 wherein the body part is a face.

21. The method of claim 19 wherein the localization step is based on a trained objective classifier.

22. The method of claim 19 wherein the extracting of input signals from the region of interest includes separating at least two channels and computing a spectral mean, median or mode of these channels for each video frame.

23. The method of claim 19 wherein the blind source separation includes detrending and/or normalizing the input signals extracted from the region of interest.

24. The method of claim 23 wherein the blind source separation incorporates independent component analysis for the separation of source signals from the detrended and normalized input signals.

25. The method of claim 19 wherein processing of the separated source signals is performed in a time window on the order of five seconds to fifteen minutes.

26. The method of claim 19 wherein processing of the one or more selected source signals includes moving average filtering and filtering to obtain the blood volume pulse.

27. The method of claim 19 wherein the physiological parameters include heart rate, respiratory rate and heart rate variability.

28. System for determining physiological parameters comprising:
a camera for capturing video of a human face to generate at least two signals; and
a computer running a program operating on the signals to determine the blood volume pulse from which other physiological parameters may be determined.
